(19) United States
(12) Patent Application Publication
(10) Pub. No.: US 2023/0372151 A1
(43) Pub. Date: Nov. 23, 2023

(54) IMPLANTABLE PUMP FOR EYE DISEASE MANAGEMENT

(71) Applicant: Twenty Twenty Therapeutics LLC, South San Francisco, CA (US)

(72) Inventors: Martin Sheridan, Half Moon Bay, CA (US); Maxim L. Armstrong, Pacifica, CA (US); Dimitri Azar, Chicago, IL (US); Benedict Costello, Piedmont, CA (US); Allister F. McGuire, Palo Alto, CA (US)

(21) Appl. No.: 18/319,874

(22) Filed: May 18, 2023

Related U.S. Application Data
(60) Provisional application No. 63/344,506, filed on May 20, 2022.

(51) Int. Cl. A61F 9/007

(52) U.S. Cl. CPC A61F 9/00781

(57) ABSTRACT

An implantable assembly for managing eye disease has a passive flow structure to drain aqueous humor, AH, and a pump coupled to the passive flow structure to force the AH through the passive flow structure. The pump is configured to be activated, to thereby accelerate drainage of the AH through the passive flow structure, only when powered directly from a wireless power transfer source that is external to the eye and that is brought into proximity with the eye. Other aspects are also described and claimed.

### CROSS-REFERENCE TO RELATED APPLICATION

This U.S. nonprovisional patent application claims the benefit of the earlier filing date of U.S. Provisional Application No. 63/344,506, filed 20 May 2022.

### FIELD

One aspect of this disclosure relates to techniques for draining a fluid that is referred to as aqueous humor from an eye of a human, in order to reduce intraocular pressure. Other aspects are also described.

### BACKGROUND

Intraocular pressure (TOP) refers to the pressure of a fluid known as the aqueous humor, AH, inside the eye. The pressure is normally regulated by changes in the production and outflow of the AH, but some persons suffer from disorders, such as glaucoma, which cause chronic heightened TOP. Over time, heightened TOP can cause damage to the eye's optical nerve, leading to loss of vision. Presently, treatment of glaucoma involves periodically administering pharmaceutical agents to the eye to decrease TOP. These drugs can be delivered by injection or eye drops.

For those persons who are not responsive to pharmaceutical treatments, there is another form of therapy in which a glaucoma drainage device is implanted into their eye. In such a device, a passive drainage tube is implanted that connects the anterior chamber of the eye to a plate that is, for example, attached between the sclera and the conjunctiva. The plate is an outflow site into which the AH can drain, thereby reducing the TOP. These, however, have had mixed success rates. As early as one year after the device has been implanted, the person's immune response can produce sufficient scarring, in a tissue bleb that is formed at the outflow site, that stops the intended drainage of the AH, leading to elevated TOP.

### SUMMARY

One aspect of the disclosure here is a technique for active drainage of the AH via an implanted assembly that could potentially prolong the efficacy of glaucoma drainage device therapy for controlling IOP. The assembly is composed of a pump that is coupled to a passive flow structure. The pump forces the AH through the passive flow structure when activated, to thereby accelerate drainage of the AH through the passive flow structure only when it is powered directly from a wireless power transfer source that is external to the person and that is brought into proximity with the eye. There is therefore no need to implant a power source for activating the pump. In one aspect, the pump when activated forces the AH through the passive flow structure at a rate (e.g., bulk fluid flow) of 0.1 microliters/minute to 100 microliters/minute, or more particularly one to ten microliters per minute, which may be sufficient to break or to prevent the formation of scarring related fibrotic adhesions in the outflow site. Operating the pump intermittently over time, during on-demand drainage intervals, may prevent scarring at the outflow site and therefore continuously maintain a pressure drop through the passive flow structure, thereby preventing elevated TOP levels.

Another aspect of the disclosure here is an implantable assembly for managing eye disease of a person, in which there is a pharmaceutical agent flow structure and a pump that is coupled to the flow structure. The pump is configured to be activated and thereby force a pharmaceutical agent through the flow structure into the eye (e.g., a dose between ten nanoLiters to one thousand nanoLiters), only when powered directly from a wireless power transfer source that is external to the person and that is brought into proximity with the eye. This enables intermittent, on-demand delivery of the pharmaceutical agent.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have advantages not specifically recited in the above summary.

### BRIEF DESCRIPTION OF THE DRAWINGS

Several aspects of the disclosure here are illustrated by way of example and not by way of limitation in the figures of the accompanying drawings in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 illustrates an example of an implantable assembly and external wireless power source in use.

FIG. 2 shows an example of the implantable assembly in cross-section in which the pump actuator has a ferromagnet and is powered directly by magnetic field interaction.

FIG. 3 shows an example of the implantable assembly in cross-section in which the pump actuator has an energy harvesting electromagnet that energizes a piezoelectric material.

### DETAILED DESCRIPTION

Several aspects of the disclosure with reference to the figures are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

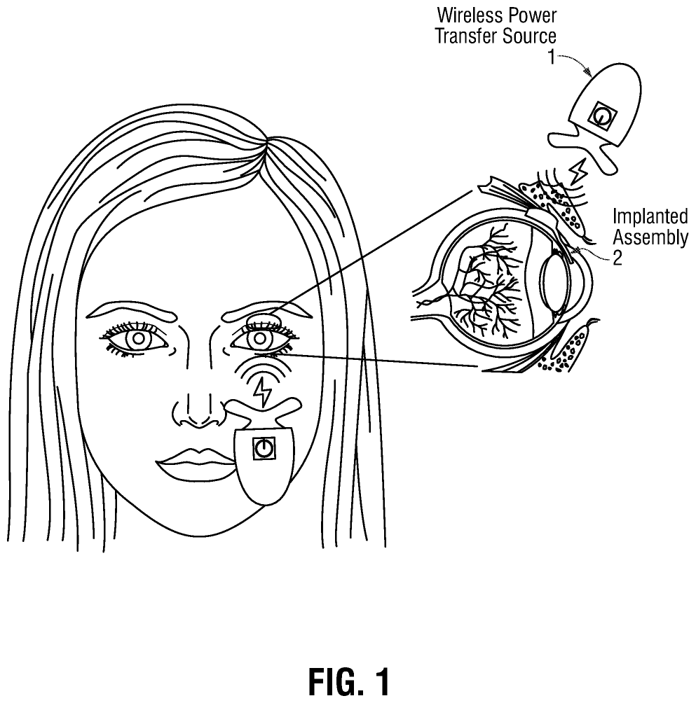

FIG. 1 illustrates an example of an implantable assembly 2 and an external wireless power transfer source 1, in use by the person into whom the assembly 2 has been surgically implanted. The implantable assembly 2 is composed of at least the following elements: a passive flow structure 3 that is implanted in the eye to drain AH from the eye into an outflow site (not shown) outside the anterior chamber of the eye; and a pump 5 that is coupled to the passive flow structure 3 to force the AH through the passive flow structure 3. The pump 5 is configured to be activated, to thereby accelerate drainage of the AH through the passive flow structure 3, only when it is being powered directly from the wireless power transfer source 1 which is external to the person, and that is brought in proximity to the eye as shown. In one aspect, the pump when activated forces the AH out of the eye through the passive flow structure and into the outflow site outside the eye at a rate (e.g., bulk fluid flow) of 0.1 microliters/minute to 100 microliters/minute, or more particularly one to ten microliters per minute. The drained AH at the outflow site may then be absorbed by the body of the person. This enables intermittent, on-demand drainage of the AH which controls the IOP of the person.

Note that the figure shows the wireless power transfer source 1 as being external to the person, such as part of a handheld device. In one instance, the housing of the power transfer source 1 could be attached to eyeglasses that can be worn by the person. For example, the power transfer source 1 could be integrated into a frame of the eyeglasses, or it could be joined to the frame in an easily removable manner. There is therefore no need to implant into the person an electrical power source such as a battery or a capacitor, for activating the pump 5.

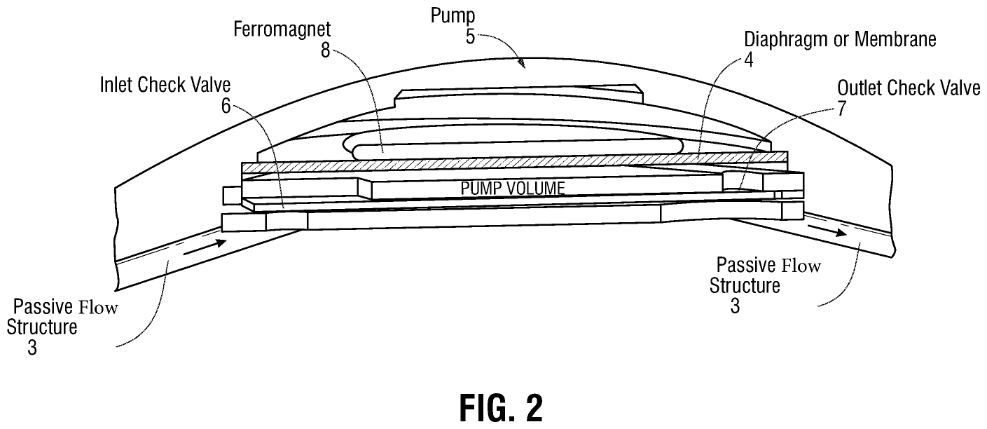

Turning now to FIG. 2, this figure shows an example of the pump 5 which may be composed of at least the following elements. A flexible membrane or diaphragm 4 has attached to it an actuator (to be described below) that is to be energized directly from and when the wireless power transfer source 1 has been brought into proximity—see FIG. 1. There is also an inlet check valve 6 and an outlet check valve 7, both of which communicate with what may be an otherwise closed, variable pump volume or variable volume that is defined in part by the flexible membrane or diaphragm 4 and in part by a substrate or frame portion to which the membrane or diaphragm 4 is attached. The inlet check vale 6 is directly coupled to an inlet portion of the passive flow structure 3 (shown to the left of the pump 5 in the figure) from which it enables fluid flow into the pump volume, while the outlet check valve 7 is directly coupled to an outlet portion of the passive flow structure 3 (shown to the right of the pump 5 in the figure) into which it enables fluid flow out of the pump volume. Each valve may be implemented as a separate flap valve.

The actuator is configured to, when energized as described further below, displace the membrane or diaphragm 4 sequentially over several cycles thereby resulting in a bulk flow of the AH, from the eye and out through the passive flow structure 3 (and into the outflow site.) There are separate ways of achieving this actuation, depending on the actuation mechanism and the type of membrane or diaphragm 4. In a first instance, the actuator when energized will drive or displace the flexible membrane or diaphragm 4 in a direction that increases the variable pump volume thereby drawing the AH into the volume through the inlet check valve 6. In a second instance, the actuator when energized will drive displace the flexible membrane or diaphragm in another direction that decreases the volume thereby forcing the AH out of the volume through the outlet check valve. In a third instance, the actuator is energized to drive or displace the flexible membrane or diaphragm in both directions to sequentially increase the volume and then decrease the volume. For example, in the case of the first instance and the second instance, the membrane or diaphragm 4 may be a diaphragm that is inherently (or otherwise) spring loaded so that it returns to a default or biased position once the pump 5 has been de-energized—in that case the pump 5 may only need to be energized in each cycle to displace the membrane or diaphragm in a single direction. In the case of the third instance, the membrane or diaphragm could be a flexible membrane and as such the pump would need to be energized in each cycle to displace the membrane in both directions sequentially.

There are various techniques for powering the pump 2, or energizing the actuator, which depend on the actuation mechanism and the way in which power is transferred from the non-implantable or external, wireless power transfer source 1 (FIG. 1.) For instance, FIG. 2 shows an actuator that has an implanted ferromagnet 8 which is attached to move as one with the membrane or diaphragm 4. In this case, the pump 2 is activated (to displace the membrane or diaphragm as discussed above) directly by magnetic field interaction between the implanted ferromagnet 8 and the wireless power transfer source 1 (and only when the wireless power transfer source 1 is energized.) In another example, the magnetic field interaction may be created when an external ferromagnet (external to the person) serving as the wireless power transfer source 1 starts to oscillate (and thereby creates a changing magnetic field.) In another example, the magnetic field interaction may be created when a (stationary) electromagnet such as a coil in the wireless power transfer source 1 is energized by an oscillating signal.

In another pump actuation technique (not shown), the pump 5 has an implanted element, e.g., an implanted electromagnet such as a coil with or without ferromagnetic material, which is attached to move as one with the membrane or diaphragm 4. Such a pump is activated by magnetic field interaction created by an oscillating external ferromagnet (in the wireless power transfer source 1.)

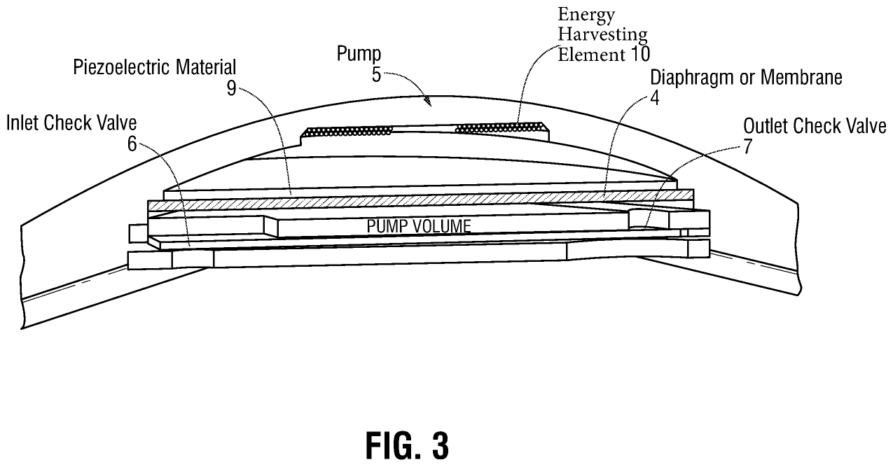

Referring now to FIG. 3, this is an example of the implantable assembly in which the pump 5 has an energy harvesting element 10, e.g., an electromagnet such as a radio frequency, RF, coil, which harvests electromagnetic energy from the wireless power transfer source 1, that applies the harvested energy to energize a piezoelectric material 9. The actuator in this case includes the piezoelectric material 9 which is attached to move as one with the membrane or diaphragm 4, and so the pump 5 is activated when the piezoelectric material 9 is energized and thereby displaces the membrane or diaphragm 4 (as described above.) The piezoelectric material may be energized by a suitable electronic driver circuit (not shown) that is responsive to a voltage change at an output of the energy harvesting element 10 which occurs whenever an external electromagnet in the wireless power transfer source 1 is energized.

In another pump actuation technique (not shown), the actuator is a thermal actuator, and the pump 5 also includes an energy harvesting element (e.g., an RF coil.) The pump 5 is activated when the thermal actuator is energized by for example a suitable electronic driver circuit that is responsive to a voltage change at the output of the energy harvesting element whenever a transmitter in the wireless power transfer source 1 (e.g., an electromagnet) is energized. The thermal actuator may be one that produces mechanical displacement because of being heated based on a thermal pneumatic effect, a shape memory alloy effect, a bimetal effect, or a mechanical thermal expansion.

In still another actuation technique (not shown), the actuator is configured to produce a phase change or pneumatic expansion that expands a gas volume, e.g., hydrolyzes water to result in oxygen or hydrogen gas, which in turn produces mechanical displacement that moves the membrane or diaphragm 4. As above, the pump is activated only when its actuator is energized by the wireless power transfer source 1.

Another aspect of the disclosure here is adapting the implantable assembly 2—see FIGS. 1-3—for delivering a pharmaceutical agent, through the passive flow structure 3 and into the eye. In this case, the left side of the passive flow structure 3 communicates with an implanted reservoir (not shown) containing a liquid pharmaceutical agent, while the right side of the passive flow structure communicates with for example the anterior chamber or other region of the eye. The pump 5 when activated draws the pharmaceutical agent and forces it through the passive flow structure 3 into the eye, only when powered directly from the wireless power transfer source (that is external to the eye and that is brought into proximity with the eye.) This enables intermittent, on-demand delivery of the pharmaceutical agent.

While certain aspects have been described above, and shown in the accompanying drawings, it is to be understood that such are merely illustrative of and not restrictive on the broad invention, and that the invention is not limited to the specific constructions and arrangements shown and described, since various other modifications may occur to those of ordinary skill in the art. The description is thus to be regarded as illustrative instead of limiting.

What is claimed is:

1. An implantable assembly for managing eye disease, the assembly comprising:
a passive flow structure to be implanted into a person to drain aqueous humor, AH, from an eye of the person; and
a pump to be implanted into the person and coupled to the passive flow structure to draw the AH from the eye and through the passive flow structure and into an outflow site, wherein the pump is configured to be activated, to thereby accelerate drainage of the AH through the passive flow structure, only when powered directly from a wireless power transfer source that is external to the eye and that is brought in to proximity with the eye.

2. The assembly of claim 1 wherein the pump when activated forces the AH through the passive flow structure at a bulk fluid flow of one to ten microliters per minute.

3. The assembly of claim 1 wherein the pump when activated forces the AH through the passive flow structure at a rate of 0.1 microliters/minute to 100 microliters/minute.

4. The assembly of claim 1 wherein no power source for activating the pump is implanted into the person.

5. The assembly of claim 1 wherein the pump comprises:
a flexible membrane or diaphragm;
an actuator attached to the flexible membrane or diaphragm;
an inlet check valve; and
an outlet check valve, wherein the inlet and outlet check valves both communicate with a variable volume that is defined in part by the flexible membrane or diaphragm,
wherein the actuator is configured to, when energized,
i) displace the flexible membrane or diaphragm to increase the volume thereby drawing the AH into the volume through the inlet check valve, or
ii) displace the flexible membrane or diaphragm to decrease the volume thereby forcing the AH out of the volume through the outlet check valve, or
iii) displace the flexible membrane or diaphragm in more than one direction to sequentially increase the volume and then decrease the volume.

6. The assembly of claim 5 wherein the actuator comprises an implanted ferromagnet, and the pump is activated by magnetic field interaction between the implanted ferromagnet and the wireless power transfer source only when the wireless power transfer source is energized.

7. The assembly of claim 5 wherein the actuator comprises an implanted electromagnet, and the pump is activated by magnetic field interaction whenever an external ferromagnet in the wireless power transfer source oscillates.

8. The assembly of claim 5 wherein the actuator comprises a piezoelectric material, an electronic driver circuit and an implanted energy harvesting element, and the pump is activated by the piezoelectric material being energized by the electronic driver circuit responsive to a voltage change at an output of the implanted energy harvesting element whenever an external electromagnet in the wireless power transfer source is energized.

9. The assembly of claim 5 wherein the actuator comprises a thermal actuator and an implanted energy harvesting element, and the pump is activated by the thermal actuator being energized responsive to a voltage change at an output of the implanted energy harvesting element whenever an external electromagnet in the wireless power transfer source is energized.

10. The assembly of claim 5 wherein the actuator comprises an implanted electromagnet and is configured to produce a phase change or pneumatic expansion that expands a gas volume that produces mechanical displacement, and the pump is activated only when the actuator is energized by a voltage change at an output of the implanted electromagnet whenever an external electromagnet in the wireless power transfer source is energized.

11. The assembly of claim 1 in combination with the wireless power transfer source being attached to eyeglasses.

12. An implantable assembly for managing eye disease, the assembly comprising:
a pharmaceutical agent passive flow structure to be implanted into an eye; and
a pump to be implanted into the eye and coupled to the pharmaceutical agent passive flow structure, wherein the pump is configured to be activated, to thereby force a pharmaceutical agent through the pharmaceutical agent passive flow structure into the eye, only when powered directly from a wireless power transfer source that is external to the eye and that is brought in to proximity with the eye.

13. The assembly of claim 12 wherein the pump when activated forces a dose of ten nanoLiters to one thousand nanoLiters of the pharmaceutical agent through the pharmaceutical agent passive flow structure.

14. The assembly of claim 12 wherein no power source for activating the pump is implanted in the eye.

15. The assembly of claim 12 wherein the pump comprises:
a flexible membrane or diaphragm;
an actuator attached to the flexible membrane or diaphragm;
an inlet check valve; and
an outlet check valve, wherein the inlet and outlet check valves both communicate with a variable volume that is defined in part by the flexible membrane or diaphragm, and the actuator when energized i) drives the flexible membrane or diaphragm to increase the variable volume thereby drawing the pharmaceutical agent into the variable volume through the inlet check valve, ii) drives the flexible membrane or diaphragm to decrease the variable volume thereby forcing the pharmaceutical agent out of the volume through the outlet check valve, or both i) and ii).

16. The assembly of claim 15 wherein the actuator comprises an implanted ferromagnet, and the pump is activated by magnetic field interaction between the implanted ferromagnet and the wireless power transfer source only when the wireless power transfer source is energized.

17. The assembly of claim 15 wherein the actuator comprises an implanted electromagnet, and the pump is activated by magnetic field interaction whenever an external ferromagnet in the wireless power transfer source oscillates.

18. The assembly of claim 15 wherein the actuator comprises a piezoelectric material, an electronic driver circuit and an implanted electromagnet, and the pump is activated by the piezoelectric material being energized by a voltage change at an output of the implanted electromagnet whenever an external electromagnet in the wireless power transfer source is energized.

19. The assembly of claim 15 wherein the actuator comprises a thermal actuator and an implanted electromagnet, and the pump is activated by the thermal actuator being energized by a voltage change at an output of the implanted electromagnet whenever an external electromagnet in the wireless power transfer source is energized.

20. The assembly of claim 15 wherein the actuator comprises an implanted electromagnet and is configured to produce a phase change or pneumatic expansion that expands a gas volume that produces mechanical displacement, and the pump is activated only when the actuator is energized by a voltage change at an output of the implanted electromagnet whenever an external electromagnet in the wireless power transfer source is energized.

21. The assembly of claim 12 in combination with the wireless power transfer source being attached to eyeglasses.
